(19) United States
(12) Patent Application Publication
(10) Pub. No.: US 2020/0171218 A1
(43) Pub. Date: Jun. 4, 2020

(54) LIQUID WASTE BAG

(71) Applicant: Chengdu Xinjin Shifeng Medical Apparatus & Instrument Co., Ltd., Chendgu City, Sichuan Province (CN)

(72) Inventors: Hejun DONG, Chengdu City, Sichuan Province (CN); Jing LI, Chengdu City, Sichuan Province (CN); Zhibiao TIAN, Nanchong City, Sichuan Province (CN); Mingyang SHI, Chengdu City, Sichuan Province (CN)

(21) Appl. No.: 16/466,234

(22) PCT Filed: Oct. 11, 2018
(86) PCT No.: PCT/CN2018/109768

§ 371 Date: Jun. 3, 2019

(30) Foreign Application Priority Data
Sep. 6, 2018 (CN) 201821456047.9

(51) Int. Cl. A61M 1/00; B65D 33/00; B65D 33/14

(52) U.S. Cl. CPC A61M 1/0019; B65D 33/14; B65D 33/004

(57) ABSTRACT

A liquid waste bag includes a bag body which is provided with a receptor cavity and a joint inserted into the receptor cavity and stuck onto the bag body. A tear part used for tearing the hag body to discharge liquid is connected to an outer wall of the receptor cavity of the bag body. The liquid waste bag is reasonable in design and simple in structure, is used to collect liquid waste and facilitates transferring and unified treatment of liquid waste. The tear part is reasonably connected to the outer wall of the receptor cavity of the bag body, the receptor cavity of the bag body can be rapidly and conveniently torn through the tear part, and then, the liquid waste is rapidly and conveniently discharged.

### BACKGROUND OF THE INVENTION

### 1. Technical Field

The invention relates to a medical consumable, in particular to a liquid waste bag.

### 2. Description of Related Art

Liquid waste bags are usually used to collect liquid waste of ICU patients and surgical patients and structurally include a white cap (or green cap), a connecting tube and a liquid waste bag body. When such liquid waste bags are used, liquid waste enters the bag body via the connecting tube, then the connecting tube is covered with the cap to prevent overflowing of the liquid waste, and afterwards, the liquid waste is transferred to a specified place to be treated in a unified manner. However, the cap needs to be unscrewed or the bag body needs to be punctured to pour the liquid waste out of the bag body, which may result in contamination and bring inconvenience to workers. Meanwhile, it is a complex procedure to cap the liquid waste bags when they are produced, and the production cost is increased.

### BRIEF SUMMARY OF THE INVENTION

To overcome the above defects, the invention provides a liquid waste bag which is reasonable in design and simple in structure, is used to collect liquid waste and facilitates transferring and unified treatment of liquid waste. According to the liquid waste bag, a tear part is reasonably connected to an outer wall of a receptor cavity of a bag body, the receptor cavity of the bag body can be rapidly and conveniently torn through the tear part, and then, liquid waste is rapidly and conveniently discharged out of the receptor cavity.

Implementation of the invention is as follows: a liquid waste bag comprises a bag body provided with a receptor cavity and a joint inserted into the receptor cavity and stuck onto the bag body. A tear part used for tearing the bag body to discharge liquid is connected to an outer wall of the receptor cavity of the bag body.

Preferably, the tear part comprises an opening strip adhering to the outer wall of the receptor cavity and a tear strip adhering to the opening strip and used for pulling the opening strip. An adhesion force of the tear strip to the opening strip is greater than an adhesion force of the opening strip to the outer wall of the receptor cavity, so that the tear strip can be easily pulled to drive the opening strip to tear the receptor cavity, and then, liquid is discharged.

Preferably, a front end of the tear strip is a free end, and an operator can pull the tear strip through the free end so as to tear the bag body more easily.

Preferably, a front end of the opening strip is narrower than a rear end of the opening strip. The width of the opening strip is gradually decreased from back to front, and a great concentrated stress is generated at the narrow front end of the opening strip, so that the operator can tear the receptor cavity more easily by pulling the opening strip, and then, liquid is discharged out of the receptor cavity more conveniently.

Preferably, the tear part is horizontally arranged on an upper portion of the receptor cavity. The tear part is arranged on the upper portion of the outer wall of the receptor cavity, so that liquid can be conveniently discharged after the receptor cavity is torn.

Preferably, the bag body is formed by high-frequency heat bonding of two films, and the receptor cavity is formed between the two films.

Preferably, a scale used for reading the liquid volume in the receptor cavity is marked on an edge of the bag body and is located between the edge of the bag body and heat-bonded strips formed by high-frequency heat bonding.

Preferably, the scale is formed by high-frequency heat bonding, so that secondary pollution caused by traditional scales printed with ink is avoided.

Preferably, hanging holes used for hanging the bag body are respectively formed in four corners of the bag body, and the bag body can be hung in multiple directions through the hanging holes.

Preferably, a plurality of heat-bonded strips are formed around the hanging holes by heat bonding, so that the strength around the hanging holes is ensured, and the bag body is prevented from being broken.

Preferably, the liquid waste bag is made from a transparent material.

The invention has the following beneficial effects:

The liquid waste bag is reasonable in design and simple in structure, is used to collect liquid waste and facilitates transferring and unified treatment of liquid waste; the tear part is reasonably connected to the outer wall of the receptor cavity of the bag body, the receptor cavity of the bag body can be rapidly and conveniently torn through the tear part, and then, liquid waste is rapidly and conveniently discharged out of the receptor cavity.

The tear part is reasonably designed by including the opening strip and the tear strip used for pulling the opening strip, so that an operator can drive the opening strip to tear the receptor cavity of the bag body only by pulling the tear strip, and then liquid waste is discharged; the opening strip is reasonably designed in that the width of the opening strip is gradually decreased from back to front, so that a great concentrated stress is formed at the narrow front end of the opening strip, the operator can tear the receptor cavity more easily by pulling the opening strip, and then liquid waste is discharged out of the receptor cavity more conveniently.

The tear strip is reasonably designed by configuring the front end of the tear strip as a free end, so that the operator can pull the tear strip through the free end so as to tear the bag body more easily, and then liquid is discharged; the scale formed by high-frequency heat bonding is reasonably designed, so that secondary pollution caused by traditional scales printed with ink is avoided; and the liquid waste bag is simple in structure, convenient to use, easy to produce, good in service effect and suitable for wide application and popularization.

### BRIEF DESCRIPTION OF THE SEVERAL VIEWS OF THE DRAWINGS

FIG. 1 is a perspective view of the invention;

FIG. 2 is a front view of the invention;

FIG. 3 is a side view of the invention;

FIG. 4 is a top view of the invention;

FIG. 5 is a partial enlarged view of the invention;

FIG. 6 is a sectional view of a tear part of the invention;

Reference signs: 1, bag body; 2, joint; 3, heat-bonded strip; 4, hanging hole; 5, tear strip; 6, opening strip; 7, liquid waste bag.

### DETAILED DESCRIPTION OF THE INVENTION

Technical solutions in embodiments of the invention are clearly and completely expounded below with reference to the accompanying drawings 1-6. Apparently, the embodiments in the following description are only illustrative ones and are not all possible ones of the invention. All other embodiments obtained by those ordinarily skilled in this field based on these illustrative ones without creative work should also fall within the protection scope of the invention.

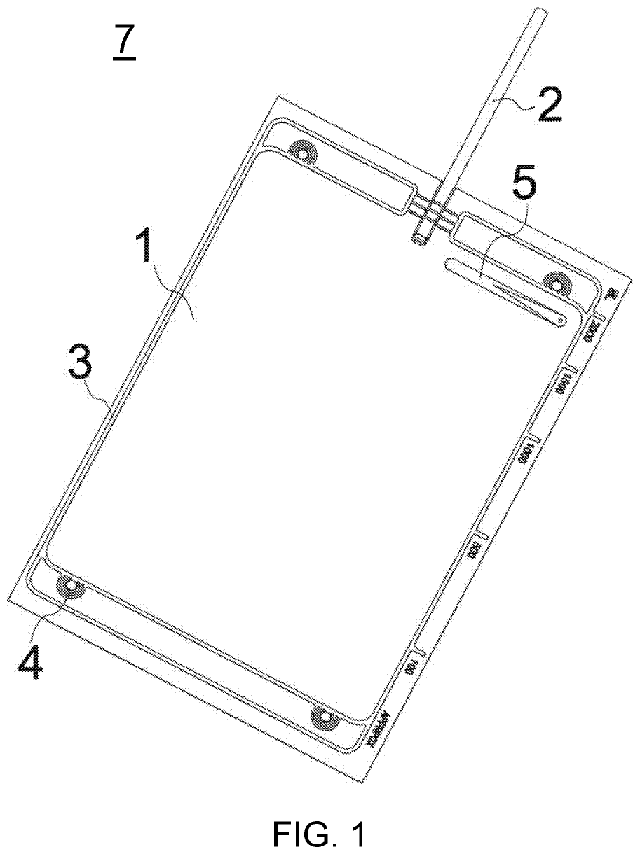

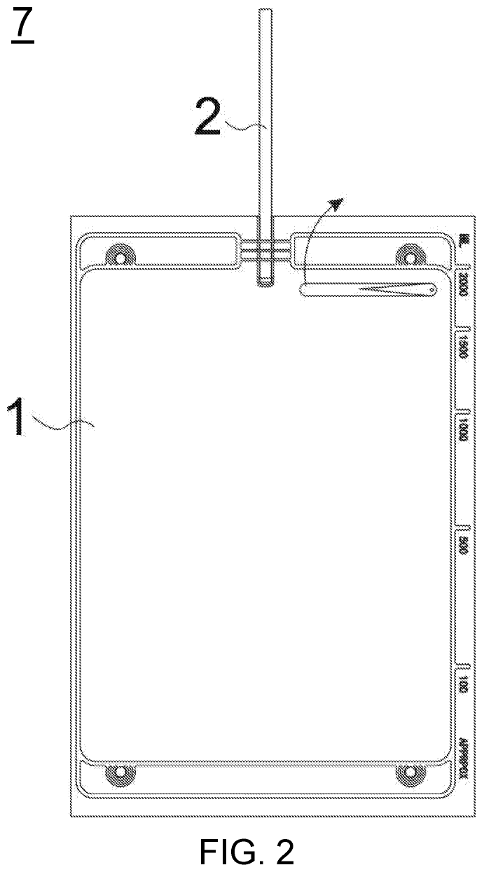

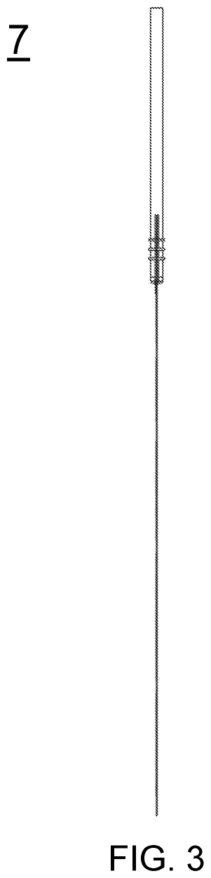

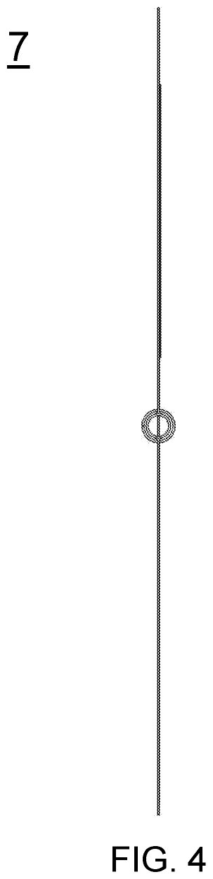

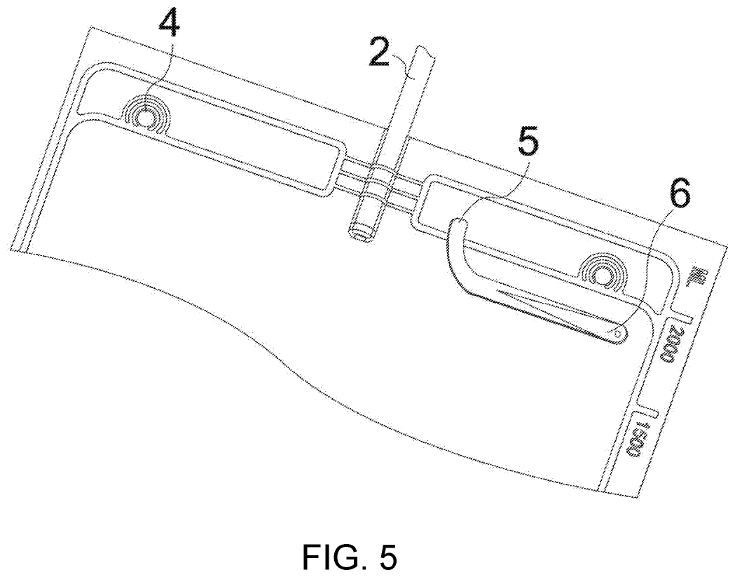

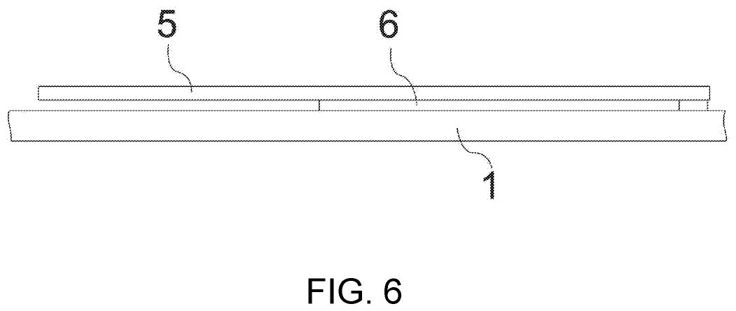

As shown in FIGS. 1-6, the invention provides a liquid waste bag 7. The liquid waste bag comprises a bag body 1 provided with a receptor cavity and a joint 2 inserted into the receptor cavity and stuck on the bag body. A tear part used for tearing the bag body to discharge liquid is connected to an outer wall of the receptor cavity of the bag body 1.

In this embodiment, the tear part comprises an opening strip 6 adhering to the outer wall of the receptor cavity and a tear strip 5 adhering to the opening strip 6 and used for pulling the opening strip 6. An adhesion force of the tear strip to the opening strip is greater than an adhesion force of the opening strip to the outer wall of the receptor cavity, so that the tear strip can be easily pulled to drive the opening strip to tear the receptor cavity, and then, liquid is discharged.

In this embodiment, a front end of the tear strip 5 is a free end, and an operator can pull the tear strip through the free end so as to tear the bag body more easily.

In this embodiment, a front end of the opening strip 6 is narrower than a rear end of the opening strip 6. The width of the opening strip is gradually decreased from back to front, and a great concentrated stress is generated at the narrow front end of the opening strip, so that the operator can tear the receptor cavity more easily by pulling the opening strip, and then, liquid is discharged out of the receptor cavity more conveniently.

In this embodiment, the tear part is horizontally arranged on an upper portion of the outer wall of the receptor cavity. The tear part is arranged on the upper portion of the outer wall of the receptor cavity, so that liquid can be conveniently discharged after the receptor cavity is torn.

In this embodiment, the bag body 1 is formed by high-frequency heat bonding of two films, and the receptor cavity used for receiving liquid is formed between the two films.

In this embodiment, a scale used for reading the liquid volume in the receptor cavity is marked on an edge of the bag body 2 and is located between the edge of the bag body and heat-bonded strips 3 formed by high-frequency heat bonding.

In this embodiment, the scale is formed by high-frequency heat bonding, so that secondary pollution caused by traditional scales printed with ink is avoided.

In this embodiment, hanging holes 4 used for hanging the bag body are respectively formed in four corners of the bag body 1, and the bag body can be hung in multiple directions through the hanging holes 4.

In this embodiment, a plurality of heat-bonded strips are formed around the hanging holes 4 by heat bonding, so that the strength around the hanging holes is ensured, and the bag body is prevented from being broken.

Specific usage of the liquid waste bag of the invention is as follows:

The liquid waste bag is connected with an external liquid waste source through the joint, and then liquid waste enters the receptor cavity of the bag body via the joint so as to be collected and treated; during liquid waste collection, the liquid waste bag can be hung in various directions through the hanging holes; after the liquid waste bag is full of liquid waste, the joint is clamped by a non-return clip to prevent overflowing of the liquid waste; afterwards, the liquid waste bag full of the liquid waste is moved to a specified position; when the liquid waste needs to be discharged out of the bag after the liquid waste bag is moved to the specified position, a worker manually pulls the tear strip, and then the opening strip is driven by the tear strip to tear the receptor cavity of the bag body, so that a liquid waste outlet is rapidly formed to discharge the liquid waste.

The liquid waste bag is reasonable in design and simple in structure, is used to collect liquid waste and facilitates transferring and unified treatment of liquid waste. The tear part is reasonably connected to the outer wall of the receptor cavity of the bag body, the receptor cavity of the bag body can be rapidly and conveniently torn through the tear part, and then, liquid waste is rapidly and conveniently discharged out of the receptor cavity.

The tear part is reasonably designed by including the opening strip and the tear strip used for pulling the opening strip, so that an operator can drive the opening strip to tear the receptor cavity of the bag body only by pulling the tear strip, and then the liquid waste is discharged. The opening strip is reasonably designed in that the width of the opening strip is gradually decreased from back to front, so that a great concentrated stress is formed at the narrow front end of the opening strip, the operator can tear the receptor cavity more easily by pulling the opening strip, and then the liquid waste is discharged out of the receptor cavity more conveniently.

Those skilled in this field can implement or use the liquid waste bag of the invention by referring to the above description of embodiments disclosed. Various modifications of these embodiments are easily achievable for those skilled in this field, and the general principle defined in the description can be fulfilled in other embodiments without deviating from the spirit or scope of the invention. Therefore, the invention is not limited to these embodiments illustrated above and should have the maximum scope in conformity with the principle and novelty disclosed by this application.

1. A liquid waste bag, comprising a bag body provided with a receptro cavity and a joint inserted into the receptor cavity and stuck onto the bag body, wherein a tear part used for tearing the bag body to discharge liquid is connected to an outer wall of the receptor cavity of the bag body.

2. The liquid waste bag according to claim 1, wherein the tear part comprises an opening strip adhering to the outer wall of the receptor cavity and a tear strip adhering to the opening strip and used for pulling the opening strip.

3. The liquid waste bag according to claim 2, wherein a front end of the tear strip is a free end.

4. The liquid waste bag according to claim 2, wherein a front end of the opening strip is narrower than a rear end of the opening strip.

5. The liquid waste bag according to claim 1, wherein the tear part is horizontally arranged on an upper portion of the receptor cavity.

6. The liquid waste bag according to claim 1, wherein the bag body is formed by high-frequency heat bonding of two films.

7. The liquid waste bag according to claim 6, wherein a scale used for reading a liquid volume in the receptor cavity is marked on an edge of the bag body and is located between the edge of the bag body and heat-bonded strips formed by high-frequency heat bonding.

8. The liquid waste bag according to claim 7, wherein the scale is formed by high-frequency heat bonding.

9. The liquid waste bag according to claim 1, wherein hanging holes used for hanging the bag body are respectively formed in four corners of the bag body.

10. The liquid waste bag according to claim 9, wherein a plurality of heat-bonded strips are formed around the hanging holes by head bonding.

11. The liquid waste bag according to claim 2, wherein the tear part is horizontally arranged on an upper portion of the receptor cavity.

12. The liquid waste bag according to claim 6, wherein hanging holes used for hanging the bag body are respectively formed in four corners of the bag body.

13. The liquid waste bag according to claim 7, wherein hanging holes used for hanging the bag body are respectively formed in four corners of the bag body.

14. The liquid waste bag according to claim 8, wherein hanging holes used for hanging the bag body are respectively formed in four corners of the bag body.

15. The liquid waste bag according to claim 12, wherein a plurality of heat-bonded strips are formed around the hanging holes by heat bonding.

16. The liquid waste bag according to claim 13, wherein a plurality of heat-bonded strips are formed around the hanging holes by heat bonding.

17. The liquid waste bag according to claim 14, wherein a plurality of heat-bonded strips are formed around the hanging holes by heat bonding.
